(19) United States
(12) Patent Application Publication
(10) Pub. No.: US 2026/0083940 A1
(43) Pub. Date: Mar. 26, 2026

(54) MEDICAL DEVICE THAT REDUCES FRICTION BETWEEN TWO CONTACTING COMPONENTS

(71) Applicant: Boston Scientific Scimed, Inc., Maple Grove, MN (US)

(72) Inventors: Andrew James Schaubhut, Bolton, MA (US); Scott E. Brechbiel, Burlington, MA (US); Ryan V. Wales, Northborough, MA (US)

(21) Appl. No.: 19/331,789

(22) Filed: Sep. 17, 2025

Related U.S. Application Data
(60) Provisional application No. 63/697,000, filed on Sep. 20, 2024.

(51) Int. Cl. A61M 25/09; A61M 25/00; A61M 25/06; A61M 39/10

(52) U.S. Cl. CPC A61M 25/09; A61M 25/0032; A61M 25/0662; A61M 39/10; A61M 2025/0037; A61M 2025/0681; A61M 2025/09058; A61M 2039/1077; A61M 2205/0222

(57) ABSTRACT

A medical device configured to create a lubricating barrier between two surfaces to reduce frictional forces, which may facilitate movement of an elongated member with respect to a device. In some examples, a medical device may include an elongated member including a sidewall defining a lumen, where the elongated member includes a perforated section with a plurality of holes that extend through the sidewall. A device may include a connector configured to connect an open end of the lumen to a fluid transfer device to receive lubricating fluid.

### CROSS-REFERENCE TO RELATED APPLICATION

This application claims priority to U.S. Provisional Patent Application No. 63/697,000, filed on Sep. 20, 2024, entitled “MEDICAL DEVICE THAT REDUCES FRICTION BETWEEN TWO CONTACTING COMPONENTS”, the disclosure of which is incorporated by reference herein in its entirety.

### TECHNICAL FIELD

This disclosure relates generally to a medical device, including an elongated member and/or a device that receives the elongated member, that can reduce friction between the elongated member and the device.

### BACKGROUND

A medical device component, such as a guidewire, may move (e.g., slide) relative to another device (e.g., an insertion device, a lumen device, a biliary device, etc.). Frictional forces between the two components may make movement of the elongated device relatively difficult.

### SUMMARY

In some aspects, the techniques described herein relate to a medical device including: an elongated member including a sidewall defining a lumen, the elongated member including a perforated section with a plurality of holes that extend through the sidewall; and a connector configured to connect an open end of the lumen to a fluid transfer device to receive lubricating fluid.

In some aspects, the techniques described herein relate to a medical device including: a device including a tube member defining a first lumen and a second lumen, the device including a plurality of holes that extend from the first lumen to the second lumen, the first lumen including a closed end and an open end, the open end configured to receive lubricating fluid from a fluid transfer device; and an elongated member configured to be inserted into the second lumen, the device configured to move lubricating fluid through the first lumen, through the plurality of holes, and into the second lumen.

In some aspects, the techniques described herein relate to a method including: creating a perforated section of an elongated member by forming a plurality of holes through a sidewall of the elongated member, the sidewall defining a lumen, the elongated member including an open end and a closed end; and coupling a connector to the open end of the elongated member, the connector configured to be coupled to a fluid transfer device to transfer fluid into the lumen and out of the plurality of holes.

In some aspects, the techniques described herein relate to a medical device including: an elongated member including a sidewall defining a lumen, the elongated member including a perforated section with a plurality of holes that extend through the sidewall; and a connector configured to connect an open end of the lumen to a fluid transfer device to receive lubricating fluid.

In some aspects, the techniques described herein relate to a medical device including: a device including a tube member defining a first lumen and a second lumen, the device including a plurality of holes that extend from the first lumen to the second lumen, the first lumen including a closed end and an open end, the open end configured to receive lubricating fluid from a fluid transfer device; and an elongated member configured to be inserted into the second lumen, the device configured to move lubricating fluid through the first lumen, through the plurality of holes, and into the second lumen.

In some aspects, the techniques described herein relate to a method including: inserting a device into a body of a patient, the device including a lumen; inserting an elongated member into the lumen of the device; and moving lubricating fluid into the lumen of the device to minimize a friction between the device and the elongated member.

### BRIEF DESCRIPTION OF THE DRAWINGS

FIG. 1A illustrates a medical device with an elongated member having a sidewall defining a lumen and a plurality of holes that expel lubricating fluid from the lumen and out of the holes according to an aspect.

FIG. 1B illustrates an example of a connector that connects the elongated member with a fluid transfer device according to an aspect.

FIG. 1C illustrates an example of the elongated member disposed within a device according to an aspect.

FIG. 1D illustrates a moveable sheath in a retracted position in which the holes are exposed according to an aspect.

FIG. 1E illustrates the moveable sheath in an extended position in which the holes are covered according to an aspect.

FIG. 2 illustrates an example of a device that can transfer lubricating fluid to a lumen that receives an elongated member according to an aspect.

FIG. 3 illustrates an example of another device that can transfer lubricating fluid to a lumen that receives an elongated member according to an aspect.

FIG. 4 illustrates a medical device with a device, an elongated member, and a motion actuator that applies motion to the elongated member to reduce friction between the device and the elongated member according to an aspect.

FIG. 5 illustrates a flowchart depicting example operations of lubricating a lumen of a device that receives an elongated member according to an aspect.

FIG. 6 illustrates a flowchart depicting example operations of reducing friction between an elongated member and a device that receives the elongated member according to an aspect.

FIG. 7 illustrates a flowchart depicting example operations of creating a medical device according to an aspect.

### DETAILED DESCRIPTION

This disclosure relates to a medical device that creates a lubricating barrier between two surfaces to reduce frictional forces, which may facilitate movement of an elongated member with respect to a device. In some examples, the devices and techniques discussed herein may avoid the use of per- and polyfluoroalkyl (PFAS) substances applied to any of components of the medical device (or one or more of the components of the medical device). For example, the medical device may enable a lubricating fluid (e.g., a non-PFAS material) (e.g., carbon dioxide (CO2), water, saline, oil (e.g., vegetable oil), etc.) to be expelled from an elongated member (e.g., a guidewire, biasing member, dilator, etc.) or a device (e.g., insertion device, biliary device (e.g., multi-lumen biliary device), scope, etc.) to create a lubricating barrier between two sliding surfaces (e.g., elongated member, device) to reduce frictional forces.

The medical device includes an elongated member that includes a sidewall defining a lumen. In some examples, the elongated member is a guidewire, biasing member, dilator, tube, or a needle, etc. The elongated member includes a plurality of holes (e.g., perforated holes) (e.g., through-holes) that extend through the sidewall of the elongated member. The medical device may include a connector (e.g., a Tuohy Borst connector with a Luer lock) that connects a proximal end portion (e.g., the proximal end portion of the lumen) of the elongated member to a fluid transfer device (e.g., a syringe, pump device, etc.).

The medical device includes a device that defines a lumen that receives the elongated member. The device's lumen may be slightly larger than the size (e.g., diameter) of the elongated member. The elongated member and the device may slide with respect to each other (e.g., the elongated member slides within a lumen of the device). In some examples, the device is an insertion device, scope device, a tubular member, a biliary device, or a multi-lumen device configured to be inserted into a body of a patient, and the elongated member may be inserted into the body via the device's lumen. The fluid transfer device may transfer lubricating fluid to the lumen of the elongated member, and then transfer the lubricating fluid radially outward from the elongated member via the holes, where the lubricating fluid creates a lubricating barrier to enable the elongated member to smoothly slide within the lumen of the device. For example, lubricating fluid may be inserted into the lumen elongated member, which is expelled radially outward from the elongated member via the holes and into the device's lumen to enable the elongated member to slide with respect to the device with less friction.

In some examples, the device is a multi-lumen device. In some examples, the multi-lumen device is a tubular member that defines multiple lumens. The device may include a first lumen and a second lumen. The first and second lumens may extend through a length of the tubular member. In some examples, the second lumen is at least partially in parallel with the first lumen. The device includes a plurality of holes that extend from the first lumen to the second lumen, where the first lumen is configured to receive and/or hold lubricating fluid. The medical device may include an elongated member configured to be inserted into the second lumen. The lubricating fluid may move through the first lumen, through the plurality of holes, and into the second lumen to create a lubricious barrier in the second lumen that receives the elongated member. In some examples, the medical device includes a connector that connects the device (e.g., the first lumen) to a fluid transfer device, and the fluid transfer device moves lubricating fluid into the first lumen and into the second lumen via the plurality of holes.

In some examples, the first lumen is an annular lumen that surrounds the first lumen. In some examples, the second lumen is a circular hole that extends a length (or a portion thereof) of the device. In some examples, the first lumen is an annular hole that forms a ring around the second lumen. In some examples, the device includes an outer sheath that defines the first lumen. Portions of the tubular member exist between the first lumen and the second lumen, but the first lumen and the second lumen are connected to each other via the holes.

In some examples, the medical device includes a motion actuator that applies motion to the elongated member to reduce friction between the device and the elongated member. The use of the motion actuator may convert a guidewire into a dynamic guidewire. The motion actuator may be a motor, vibration exciter, a linear actuator, or other mechanized device that may be attached to a guidewire entrance pout outside of a scope to allow for motion to be applied to the elongated member while the elongated member is sent through the device. The motion may be rotation, vibration (e.g., high frequency vibration), back and forth movement (e.g., continuous back and forth movement), and/or a combination of these movements.

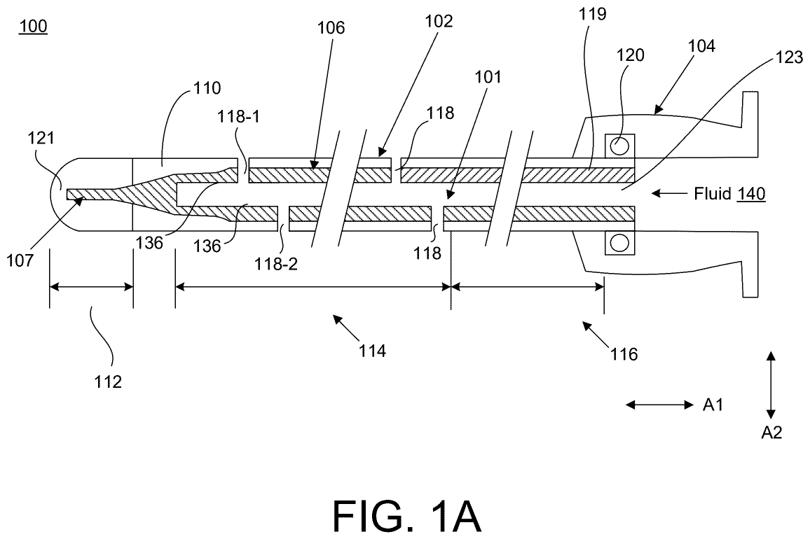

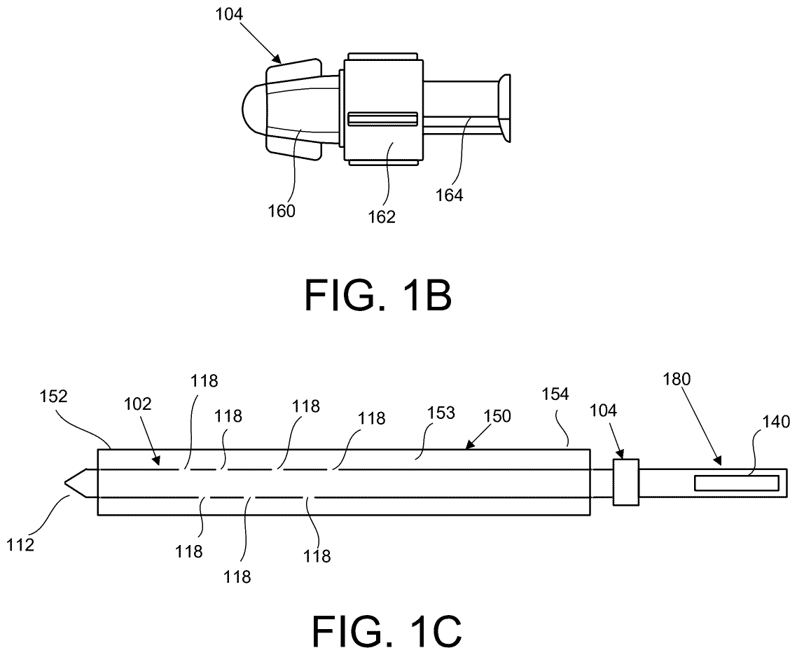

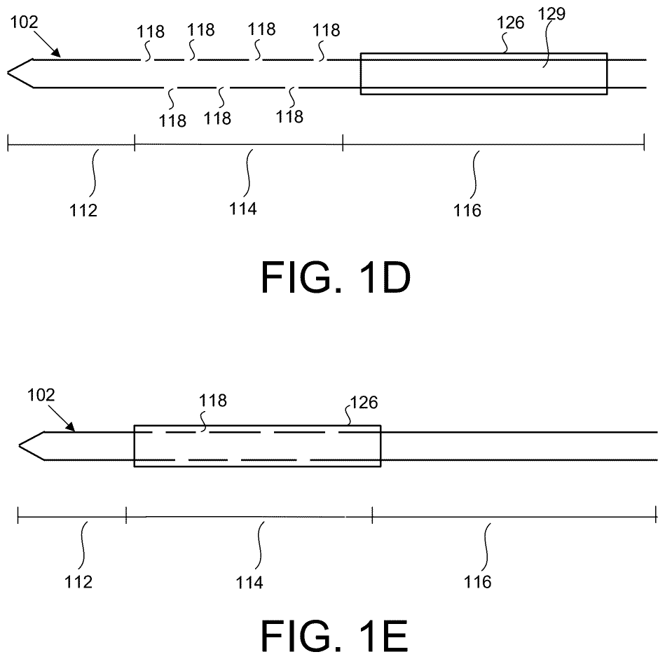

FIGS. 1A to 1E illustrate an example of a medical device 100 that creates a lubricious barrier between two surfaces to minimize frictional forces, which may facilitate movement (e.g., sliding) of an elongated member 102 with respect to a device 150. In some examples, the medical device 100 may avoid the use of PFAS substances. For example, the medical device 100 may enable a lubricating fluid 140 (e.g., CO2, water/saline, vegetable oil, etc.) to be expelled from an elongated member 102 to create a lubricating barrier between two sliding surfaces (e.g., elongated member 102, device 150) to reduce frictional forces. In some examples, the lubricating fluid 140 is any non-PFAS material. In some examples, the lubricating fluid 140 is carbon dioxide (CO2), water, saline, oil (e.g., vegetable oil), or any type of fluid. In some examples, the lubricating fluid 140 is a non-liquid material such as a lubricating gas.

As shown in FIG. 1A, the medical device 100 includes an elongated member 102. The elongated member 102 may have a tubular structure. The elongated member 102 is a guidewire (e.g., a medical guidewire). A guidewire may be a flexible wire used to navigate and access the inside of the body during a minimally invasive medical procedure. In some examples, the elongated member 102 is a biasing member. In some examples, the elongated member 102 is a dilator. In some examples, the elongated member 102 is a tube. The elongated member 102 may include a tube member 106 and a protective layer 110 on a surface of the tube member 106. In some examples, the protective layer 110 includes a polymer layer. In some examples, the protective layer 110 is referred to as a polymer coating. The tube member 106 may be a cylindrical structure. In some examples, the tube member 106 includes an alloy material. In some examples, the tube member 106 includes a shape memory material. The tube member 106 may include a metallic material. In some examples, the tube member 106 includes a Nitinol material (e.g., also referred to as a Nitinol tube).

The elongated member 102 may include a hollow center that extends at least partially along a length of the elongated member 102. For example, the elongated member 102 includes a sidewall 136 defining a lumen 101. In some examples, the lumen 101 has a circular cross-section. The sidewall 136 may be a cylindrical surface that forms the body of the tube. The thickness of the sidewall 136 may be defined as the distance between an inner surface and an outer surface of the sidewall 136. The lumen 101 extends through the elongated member 102 in the direction A1. In some examples, the direction A1 is aligned with a center line of the lumen 101. The lumen 101 has an open end 123 at a proximal end of the elongated member 102. The lumen 101 may have a closed end 107 at a distal end of the elongated member 102.

The elongated member 102 includes a plurality of holes 118. The holes 118 extend through (e.g., entirely through) the sidewall 136. The holes 118 may extend entirely through the tube member 106 and the protective layer 110. In some examples, the holes 118 have a circular cross-section. In some examples, the holes 118 have a square cross-section. In some examples, the holes 118 extend radially outward from the lumen 101. For example, the holes 118 may extend in the direction A2, where the direction A2 is perpendicular or orthogonal to the direction A1. A lubricating fluid 140 may be expelled from the lumen 101 and out of the holes 118 to create a lubricating barrier when the elongated member 102 slides within a lumen 153 of a device 150.

The elongated member 102 includes a distal end portion 112, a perforated section 114, and a non-perforated section 116. In some examples, the non-perforated section 116 comprises the majority of the length of the elongated member 102 in the direction A1. In some examples, the distal end portion 112 includes a solid tip portion (e.g., a hydrophilic tip) (e.g., a solid hydrophilic tip). In some examples, the distal end portion 112 is devoid of a lumen. For example, the distal end portion 112 includes a portion of the tube member 106 that is devoid of a lumen 101 (e.g., a solid metallic (e.g., alloy) portion). In some examples, the distal end portion 112 includes a protective layer portion that is thicker than the protective layer 110 at the perforated section 114 in the direction A2. At the distal end portion 112, the tube member 106 may have a diameter that is less than the tube member 106 at the perforated section 114 and the non-perforated section 116. In some examples, although the tube member 106 includes a varying thickness in the direction A2 (e.g., due to the lack of lumen at the distal end portion 112), the protective layer 110 that is disposed over the outer surface of the tube member 106 provides a relatively uniform thickness in the direction A2. In some examples, the distal end portion 112 includes a rounded tip 121.

The perforated section 114 defines the plurality of holes 118. In some examples, the holes 118 are uniformly spaced apart along a length of the perforated section 114. In some examples, the holes 118 have a higher density at a distal portion of the perforated section 114 as opposed to a proximal portion of the perforated section 114. In some examples, the holes 118 are not disposed on opposite sides on the sidewall 136. For example, a location of a hole 118-1 (on one side of the sidewall 136) is offset from a location of a hole 118-2 (on the opposite side of the sidewall 136) in a direction A1. In some examples, the perforated section 114 has a length that is greater than the length of the distal end portion 112 in the direction A1. In some examples, the length of the distal end portion 112 is between 3 centimeters and 15 centimeters. In some examples, the length of the distal end portion 112 is between 5 centimeters and 10 centimeters. The perforated section 114 is located between the distal end portion 112 and the non-perforated section 116. The non-perforated section 116 is a section of the elongated member 102 that is devoid of holes 118. In some examples, the non-perforated section 116 includes a proximal end portion of the elongated member 102. In other words, the perforated section 114 is located between the distal end portion 112 of the elongated member 102 and the proximal end portion of the elongated member 102. In some examples, the non-perforated section 116 has a length that is greater than the length of the perforated section 114.

The medical device 100 includes a connector 104 that connects the elongated member 102 to a fluid transfer device 180. The connector 104 may be connected to a proximal end portion 119 of the elongated member 102. The fluid transfer device 180 is configured to transfer lubricating fluid 140 to the lumen 101 of the elongated member 102. In some examples, the connector 104 includes a Tuohy Borst connector. In some examples, the connector 104 includes a Tuohy Borst connector with a Luer lock. In some examples, the Luer lock is a female Luer lock. The connector 104 may include a ring member 120. In some examples, the ring member 120 includes a circular seal member. In some examples, the ring member 120 includes an O ring (e.g., an O ring seal). As shown in FIG. 1B, the connector 104 may include a body 160 that includes a valve configured to reduce or prevent the backflow of fluids, a rotatable member 162, and a threaded receptacle 164 that receives a corresponding connector (e.g., a male Luer lock) on the fluid transfer device 180. In some examples, the fluid transfer device 180 is a syringe device. In some examples, the fluid transfer device 180 is a pump device (e.g., an electronic pump or a manual pump).

For example, by activating the fluid transfer device 180, the lubricating fluid 140 moves through the lumen 101 and out of the plurality of holes 118 to create a lubricating barrier between the device 150 and the elongated member 102. In some examples, as shown in FIG. 1C, the elongated member 102 may move with respect to a device 150. In some examples, the device 150 includes an insertion device configured to be inserted into the body of a patient. The device 150 includes a distal end portion 152 and a proximal end portion 154. The device 150 includes a lumen 153 that extends between the distal end portion 152 and the proximal end portion 154. In some examples, the distal end portion 152 defines an open end, and the proximal end portion 154 defines an open end. In some examples, the distal end portion 112 of the elongated member 102 can distally extend out of the open end on the distal end portion 152. In some examples, the elongated member 102 has a length that is greater than the length of the device 150.

In some examples, the lumen 153 is a size (e.g., a diameter) that is slightly larger than a size (e.g., a diameter) of the elongated member 102, where the elongated member 102 can be inserted into the lumen 153 of the device 150. In order to reduce the friction between the outer surface of the elongated member 102 and the device's surface that defines the lumen 153 when the elongated member 102 is inserted into the device's lumen 153, the fluid transfer device 180 can be activated to transfer the lubricating fluid 140 into the lumen 101 and out of the elongated member 102 via the holes 118, thereby into the lumen 153 so that a lubricous barrier exists between the sliding surfaces.

In some examples, as shown in FIGS. 1D and 1E, the medical device 100 includes a retractable sleeve 126 over the elongated member 102. The retractable sleeve 126 may slide between the perforated section 114 and the non-perforated section 116. In some examples, the retractable sleeve 126 has a length that corresponds to the length of the perforated section 114. In some examples, the retractable sleeve 126 has a length that is greater than the length of the perforated section 114. The retractable sleeve 126 includes a lumen 129, where at least a portion of the elongated member 102 is disposed within the lumen 129 of the retractable sleeve 126. The retractable sleeve 126 is configured to move between a proximal position, as shown in FIG. 1D, to a distal position, as shown in FIG. 1E. In the proximal position, the retractable sleeve 126 is positioned over the non-perforated section 116, where the holes 118 are not covered by the retractable sleeve 126. In the distal position, the retractable sleeve 126 is positioned over the perforated section 114, where the holes 118 are covered by the retractable sleeve 126. In the distal position, lubricating fluid 140 is prevented from being expelled from the elongated member 102.

In some examples, the medical device 100 includes a guidewire (e.g., the elongated member 102) with a removable Luer connector (e.g., connector 104) at the proximal end, which may allow for a connection (e.g., a direct hookup) with lubricating fluid to be pumped through the middle of the guidewire. The guidewire may be constructed from a Nitinol tube with a hollow center. The guidewire includes circumferential holes (e.g., holes 118) along the length where the device 150 (e.g., tome or other accessory biliary device) is in contact with the guidewire. The lubricating fluid 140 can be controlled via a fluid transfer device 180 (e.g., an external pump or a syringe). The Luer connection to seal around the guidewire may allow the lubricating fluid 140 to flow within the center of the metal tube, which can be removed for device exchanges. In some examples, the medical device 100 includes a designed O-Ring Seal or an adjustable Tuohy Borst connector with female Luer lock. In some examples, only the middle portion of the guidewire (e.g., excluding a solid tip portion (e.g., a solid hydrophilic tip) and the portion that would normally extend out of the scope/device). In some examples, in order to reduce leakage outside a duodenoscope, a retractable sleeve 126 (e.g., an additional Nitinol or SS tube) may be added to the outside of the proximal end of the tube to cover or block some of the proximal holes extending outside of the scope, thereby minimizing leaking outside the endoscope where the user would be manipulating devices.

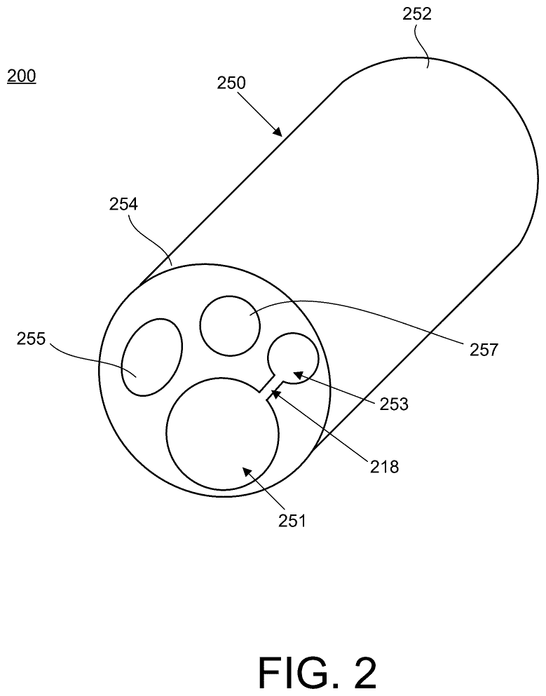

FIG. 2 illustrates an example of a medical device 200 having a device 250 that can transfer lubricating fluid to a lumen 251 that receives an elongated member (e.g., a guidewire) according to an aspect. The medical device 200 may be an example of the medical device 100 of FIGS. 1A to 1E, and, therefore, may include any of the details discussed with reference to those figures. The medical device 200 creates a lubricious barrier between two surfaces to minimize frictional forces, which may facilitate movement of an elongated member with respect to the device 250. In some examples, the medical device 200 may avoid the use of PFAS substances on the device 250 or the elongated member. The medical device 200 may enable a lubricating fluid (e.g., CO2, water/saline, vegetable oil, etc.) to be expelled from the device 250 (e.g., insertion device, biliary device (e.g., multi-lumen biliary device)) to create a lubricating barrier between two sliding surfaces (e.g., elongated member, device 250) to reduce frictional forces.

The device 250 may be a multi-lumen device. In some examples, the device 250 is a tubular member (e.g., a solid body) (e.g., with a circular cross-section) that defines multiple lumens. In some examples, the device 250 is a biliary device. The device 250 includes a distal end portion 252 and a proximal end portion 254. The device 250 includes a length that extends from the proximal end portion 254 and the distal end portion 252.

The device 250 includes a lumen 251 and a lumen 253. The lumen 251 may extend through a length of the device 250, e.g., to and through the proximal end portion 254 and the distal end portion 252. The lumen 253 may extend through a length of the device 250, e.g., to and through the proximal end portion 254 and the distal end portion 252. The lumen 251 may receive an elongated member such as the elongated member 102 of FIGS. 1A to 1E. The elongated member may be a guidewire. In some examples, the elongated member, received by the lumen 251, is different from the elongated member 102 of FIGS. 1A to 1E. The lumen 253 may receive and hold lubricating fluid. In some examples, the lumen 251 has a size (e.g., a diameter) that is greater than the size (e.g., diameter) of the lumen 253. In some examples, the lumen 251 and the lumen 253 have a circular cross-section. In some examples, the lumen 251 and the lumen 253 are parallel to each other. The device 250 includes a plurality of holes 218 that extend that connect the lumen 253 with the lumen 251.

In some examples, the medical device 200 includes a connector (e.g., the connector 104 of FIGS. 1A to 1E) that connects the device 250 (e.g., the lumen 253) to a fluid transfer device, and the fluid transfer device moves lubricating fluid into the lumen 253 and then into the lumen 251 via the plurality of holes 218, thereby creating a lubricating barrier between two sliding surfaces (e.g., elongated member, device 250) to reduce frictional forces.

In some examples, the device 250 includes a lumen 255 and a lumen 257. Although four lumens are illustrated in FIG. 2, the device 250 may include any number of lumens such as two lumens (e.g., lumen 251, lumen 253), three lumens, or any number of lumens greater than four. The lumen 255 may extend through a length of the device 250, e.g., to and through the proximal end portion 254 and the distal end portion 252. The lumen 257 may extend through a length of the device 250, e.g., to and through the proximal end portion 254 and the distal end portion 252. The lumen 255 and the lumen 257 may be parallel to each other. In some examples, the lumen 251, the lumen 253, the lumen 255, and the lumen 257 are parallel to each other.

In some examples, the medical device 200 may allow the guidewire channel (e.g., lumen 251) to have perforations to expel the lubricating fluid around the guidewire. In some examples, the medical device 200 includes an additional lumen (e.g., lumen 253) (e.g., as compared to a conventional multi-lumen biliary device) that carries a dedicated lubricating fluid (e.g., CO2, water/saline, vegetable oil), or it could be combined with the traditional injection lumen used for saline and radiopaque contrast. Small holes (e.g., holes 218) (e.g., circular or other shaped) may connect the lubrication lumen (e.g., lumen 253) to the guidewire lumen (e.g., lumen 251) along a discrete length.

A Luer lock (or similar connection) may allow syringe injection or an external pump to administer the lubricating fluid during the time beneficial for guidewire movement. In some examples, a needle that can be directed eccentrically to pierce multiple holes (e.g., holes 218) along the length of the guidewire lumen (e.g., lumen 251).

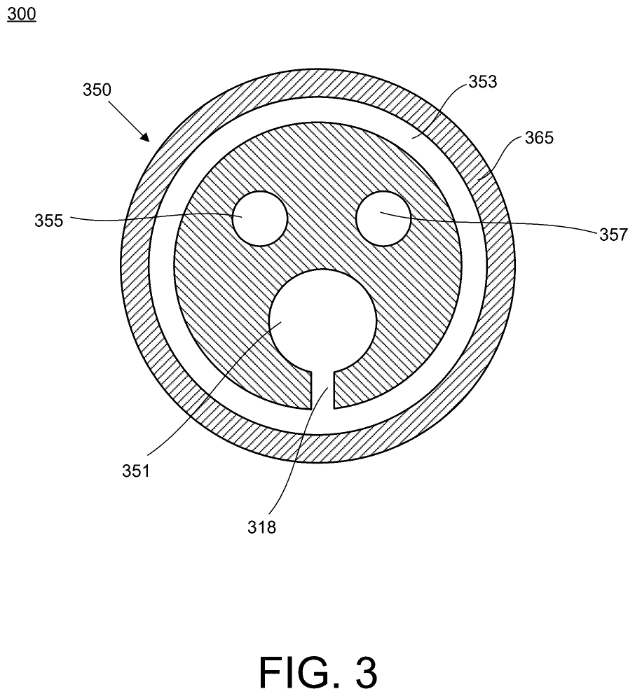

FIG. 3 illustrates an example of a medical device 300 having a device 350 that can transfer lubricating fluid to a lumen 351 that receives an elongated member (e.g., a guidewire) according to an aspect. The medical device 300 may be an example of the medical device 100 of FIGS. 1A to 1E and/or the medical device 200 of FIG. 2, and, therefore, may include any of the details discussed with reference to those figures. In some examples, the techniques discussed herein may send the holes 318 from the guidewire lumen (e.g., lumen 351) to the external side of the extrusion and then have an outer sleeve (e.g., outer sheath 365) over the device 350, thereby allowing the lubricating fluid to be transferred down the length of the device 350 through the annulus (e.g., lumen 353) created from the main extrusion and outer sheath 365.

The medical device 300 of FIG. 3 may include a lumen 353 (e.g., fluid lumen) configured as an annulus lumen that surrounds a lumen 351 that receives an elongated member (e.g., a guidewire). The medical device 300 creates a lubricious barrier between two surfaces to minimize frictional forces, which may facilitate movement of an elongated member with respect to the device 350. In some examples, the medical device 300 may avoid the use of PFAS substances on the device 350 or the elongated member. The medical device 300 may enable a lubricating fluid (e.g., CO2, water/saline, vegetable oil, etc.) to be inserted into a lumen 351 (e.g., device lumen) to create a lubricating barrier between two sliding surfaces (e.g., elongated member, device 250) to reduce frictional forces.

The device 350 may be a multi-lumen device. In some examples, the device 350 is a tubular member (e.g., a solid body) (e.g., with a circular cross-section) that defines multiple lumens. In some examples, the device 350 is a biliary device. The device 350 includes a distal end portion and a proximal end portion. The device 350 includes a length that extends from the proximal end portion and the distal end portion.

The device 350 includes a lumen 351 and a lumen 353. The lumen 351 may extend through a length of the device 350, e.g., to and through the proximal end portion and the distal end portion. The lumen 351 may receive an elongated member such as the elongated member 102 of FIGS. 1A to 1E. The elongated member may be a guidewire. In some examples, the elongated member, received by the lumen 351, is different from the elongated member 102 of FIGS. 1A to 1E. In some examples, the lumen 351 includes a straight (e.g., linear) portion.

The lumen 353 may be angular lumen that surrounds the lumen 351. In some examples, the lumen 353 has a circular shape. In some examples, the device 350 includes an outer sheath 365 with an inner surface that defines the lumen 353. The lumen 353 may have a size (e.g., a diameter) that is larger than the size (e.g., diameter) of the lumen 351. The lumen 353 may receive and hold lubricating fluid. The device 350 includes a plurality of holes 318 that extend that connect the lumen 353 with the lumen 351. In some examples, the medical device 300 includes a connector (e.g., the connector 104 of FIGS. 1A to 1E) that connects the device 350 (e.g., the lumen 353) to a fluid transfer device, and the fluid transfer device moves lubricating fluid into the lumen 353 and then into the lumen 351 via the plurality of holes 318, thereby creating a lubricating barrier between two sliding surfaces (e.g., elongated member, device 350) to reduce frictional forces.

In some examples, the device 350 includes a lumen 355 and a lumen 357. In some examples, the lumen 353 surrounds the lumen 355 and the lumen 357. In some examples, the lumen 355 and the lumen 357 are linear lumens. Although four lumens are illustrated in FIG. 3 (e.g., three linear lumens, and one angular lumen), the device 350 may include any number of lumens such as two lumens (e.g., lumen 351, lumen 353), three lumens, or any number of lumens greater than four. The lumen 355 may extend (e.g., linearly extend) through a length of the device 350, e.g., to and through the proximal end portion and the distal end portion. The lumen 357 may extend through a length of the device 350, e.g., to and through the proximal end portion and the distal end portion. The lumen 355 and the lumen 357 may be parallel to each other. In some examples, the lumen 351, the lumen 355, and the lumen 357 are parallel to each other.

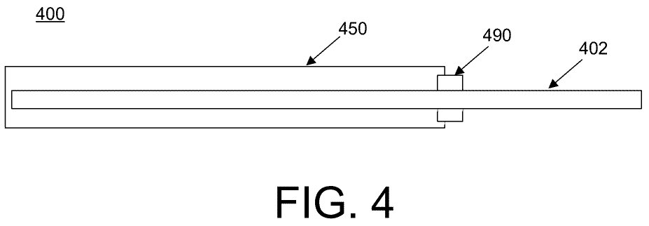

FIG. 4 illustrates a medical device 400 with a device 450, an elongated member 402, and a motion actuator 490 that applies motion to the elongated member 402 to reduce friction between the device 450 and the elongated member 402 according to an aspect. The medical device 400 may be an example of the medical device 100 of FIGS. 1A to 1E, the medical device 200 of FIG. 2, and the medical device 300 of FIG. 3, and, therefore, may include any of the details discussed with reference to those figures. In some examples, the medical device 400 may avoid the use of PFAS substances on the device 450 or the elongated member 402 but may reduce friction between two sliding surfaces by applying motion to the elongated member 402.

In some examples, the medical device 400 creates a dynamic guidewire (e.g., a dynamic elongated member 402). Rotation, high frequency vibration, continuous back and forth movement, or a combination of these options, may be be employed to encourage the lower dynamic friction coefficients between the guidewire (e.g., the elongated member 402) and accessory device (e.g., the device 450), as compared to higher static friction coefficients. A motor, vibration exciter, linear actuator or other mechanized device (e.g., motion actuator 490) may be be attached to the guidewire entrance port outside of the scope, which may allow for motion (e.g., continuous motion) to be applied while the guidewire (e.g., elongated member 402) is being sent down the biliary device or to case the biliary device being removed from the guidewire.

The medical device 400 includes a motion actuator 490 that applies motion to the elongated member 402 to reduce friction between the device 450 and the elongated member 402. The use of the motion actuator 490 may convert the elongated member 402 (e.g., a guidewire) into a dynamic elongated member (e.g., a dynamic guidewire). The motion actuator 490 may be a motor, vibration exciter, a linear actuator, or other mechanized device. In some examples, the motion actuator 490 may be coupled to a proximal end portion of the device 450. In some examples, the motion actuator 490 may be coupled to the device 450 at a location that is proximate to the guidewire opening. In some examples, the motion actuator 490 may be coupled to a guidewire entrance port outside of the device 450 (e.g., scope) to allow for motion to be applied to the elongated member 402 while the elongated member 402 is sent through the device 450. The motion may be rotation, vibration (e.g., high frequency vibration), back and forth movement (e.g., continuous back and forth movement), and/or a combination of these movements.

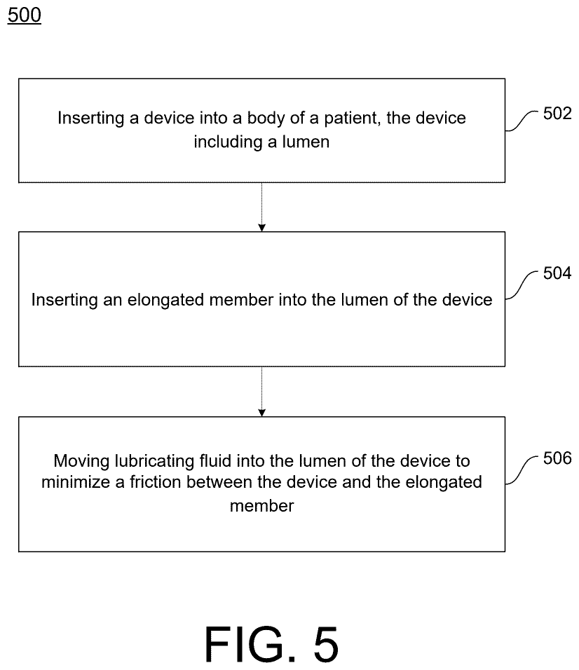

FIG. 5 illustrates a flowchart 500 depicting example operations of lubricating a lumen of a device that receives an elongated member according to an aspect. The operations of FIG. 5 create a lubricious barrier between two surfaces to minimize frictional forces, which may facilitate movement of an elongated member (e.g., a guidewire, biasing member, dilator, tube, etc.) with respect to a device (e.g., an insertion device, biliary device (e.g., multi-lumen biliary device), a scope, etc.). In some examples, the operations discussed herein may avoid the use of PFAS substances applied to any components of the medical device (e.g., the medical device is completely devoid of PFAS substances). The operations discussed herein may enable a lubricating fluid (e.g., CO2, water/saline, vegetable oil, etc.) to be expelled from an elongated member (e.g., a guidewire, biasing member, tube, or generally any insertable device) or a device (e.g., insertion device, biliary device (e.g., multi-lumen biliary device)) to create a lubricating barrier between two sliding surfaces (e.g., elongated member, device) to reduce frictional forces.

Although the flowchart 500 of FIG. 5 illustrates the operations in sequential order, it will be appreciated that this is merely an example, and that additional or alternative operations may be included. Further, operations of FIG. 5 and related operations may be executed in a different order than that shown, or in a parallel or overlapping fashion. The flowchart 500 may be applicable to any of the medical devices described herein, including medical device 100 of FIGS. 1A to 1E, medical device 200 of FIG. 2, and/or medical device 300 of FIG. 3.

Operation 502 includes inserting a device into the body of a patient, the device including a lumen. Operation 504 includes inserting an elongated member into the lumen of the device. Operation 506 includes moving lubricating fluid into the lumen of the device to minimize a friction between the device and the elongated member.

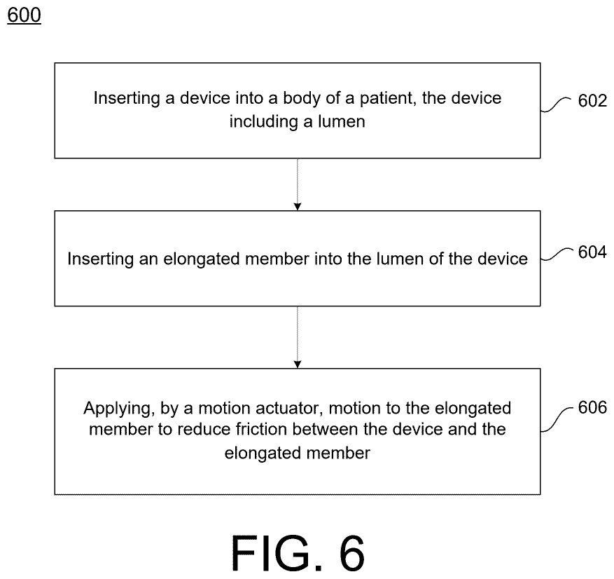

FIG. 6 illustrates a flowchart 600 depicting example operations of reducing friction between an elongated member and a device that receives the elongated member according to an aspect. In some examples, the operations of FIG. 6 may avoid the use of PFAS substances on any of the components of the medical device but may still reduce friction between two sliding surfaces by applying motion to the elongated member. Although the operations of FIG. 6 may be applied to the medical device 400 of FIG. 4, the operations of FIG. 6 may be applied to any of the implementations discussed herein, including medical device 100 of FIGS. 1A to 1E, medical device 200 of FIG. 2, and/or medical device 300 of FIG. 3. Although the flowchart 600 of FIG. 6 illustrates the operations in sequential order, it will be appreciated that this is merely an example, and that additional or alternative operations may be included. Further, operations of FIG. 6 and related operations may be executed in a different order than that shown, or in a parallel or overlapping fashion.

Operation 602 includes inserting a device into the body of a patient, the device including a lumen. Operation 604 includes inserting an elongated member into the lumen of the device. Operation 606 includes applying, by a motion actuator, motion to the elongated member to reduce friction between the device and the elongated member.

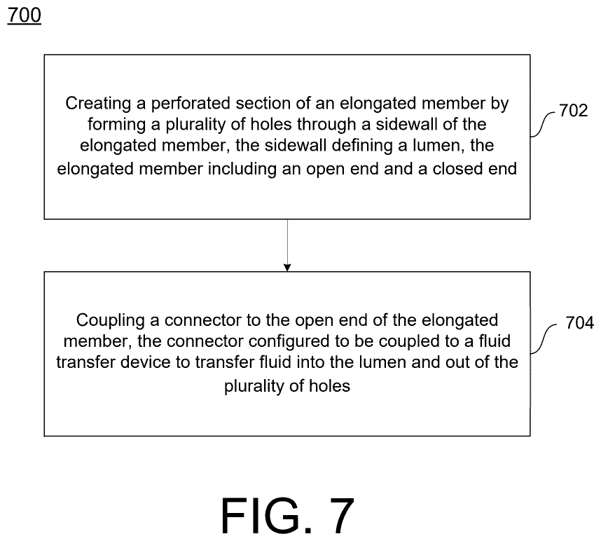

FIG. 7 illustrates a flowchart 700 depicting example operations of creating a medical device that can lubricate a lumen of a device that receives an elongated member according to an aspect. The operations of FIG. 7 create a medical device with a lubricious barrier between two surfaces to minimize frictional forces, which may facilitate movement of an elongated member (e.g., a guidewire, biasing member, dilator, tube, etc.) with respect to a device (e.g., an insertion device, biliary device (e.g., multi-lumen biliary device), a scope, etc.). In some examples, the operations discussed herein may avoid the use of PFAS substances applied to any components of the medical device (e.g., the medical device is completely devoid of PFAS substances). The operations discussed herein may enable a lubricating fluid (e.g., CO2, water/saline, vegetable oil, etc.) to be expelled from an elongated member (e.g., a guidewire, biasing member, tube, or generally any insertable device) or a device (e.g., insertion device, biliary device (e.g., multi-lumen biliary device)) to create a lubricating barrier between two sliding surfaces (e.g., elongated member, device) to reduce frictional forces.

Although the flowchart 700 of FIG. 7 illustrates the operations in sequential order, it will be appreciated that this is merely an example, and that additional or alternative operations may be included. Further, operations of FIG. 7 and related operations may be executed in a different order than that shown, or in a parallel or overlapping fashion. The flowchart 700 may be applicable to any of the medical devices described herein, including medical device 100 of FIGS. 1A to 1E, medical device 200 of FIG. 2, and/or medical device 300 of FIG. 3.

Operation 702 includes creating a perforated section of an elongated member by forming a plurality of holes through a sidewall of the elongated member, the sidewall defining a lumen, the elongated member including an open end and a closed end. Operation 704 includes coupling a connector to the open end of the elongated member, the connector configured to be coupled to a fluid transfer device to transfer fluid into the lumen and out of the plurality of holes.
Clause 1. A medical device comprising: an elongated member including a sidewall defining a lumen, the elongated member including a perforated section with a plurality of holes that extend through the sidewall; and a connector configured to connect an open end of the lumen to a fluid transfer device to receive lubricating fluid.
Clause 2. The medical device of clause 1, wherein the elongated member includes a distal end portion and a non-perforated section, the non-perforated section including a proximal end portion of the elongated member, the perforated section being disposed between the distal end portion and the non-perforated section, the perforated section including the plurality of holes.
Clause 3. The medical device of clause 2, wherein the distal end portion includes a solid tip portion defining a closed end of the lumen, the proximal end portion including the open end of the lumen.
Clause 4. The medical device of clause 2, wherein the non-perforated section comprises a majority of a length of the elongated member.
Clause 5. The medical device of any of clauses 1 to 4, wherein the elongated member includes a tube member and a protective layer on a surface of the tube member, the plurality of holes extending through the tube member and the protective layer.
Clause 6. The medical device of any of clauses 1 to 5, wherein the elongated member includes a guidewire, a biasing member, or a dilator.
Clause 7. The medical device of any of clauses 1 to 6, wherein the connector includes a ring member, the connector including a Tuohy Borst connector with a threaded receptacle.
Clause 8. The medical device of any of clauses 1 to 7, further comprising: a retractable sleeve including a lumen, at least a portion of the elongated member being disposed within the lumen of the retractable sleeve, the retractable sleeve configured to move from a proximal position on the elongated member to a distal position on the elongated member, the retractable sleeve, in the proximal position, not covering the plurality of holes, the retractable sleeve, in the distal position, covering at least a portion of the plurality of holes.
Clause 9. The medical device of any of clauses 1 to 8, further comprising: a device configured to receive the elongated member, the lubricating fluid minimizing a friction between the device and the elongated member.
Clause 10. A medical device comprising: a device including a tube member defining a first lumen and a second lumen, the device including a plurality of holes that extend from the first lumen to the second lumen, the first lumen including a closed end and an open end, the open end configured to receive lubricating fluid from a fluid transfer device; and an elongated member configured to be inserted into the second lumen, the device configured to move lubricating fluid through the first lumen, through the plurality of holes, and into the second lumen.
Clause 11. The medical device of clause 10, wherein the device includes a proximal end portion, the medical device further comprising: a connector configured to be connected to the open end of the first lumen on the proximal end portion of the device.
Clause 12. The medical device of clause 10 or 11, wherein the device includes a third lumen and a fourth lumen, wherein the first lumen and the second lumen are parallel to each other.
Clause 13. A method comprising: creating a perforated section of an elongated member by forming a plurality of holes through a sidewall of the elongated member, the sidewall defining a lumen, the elongated member including an open end and a closed end; and coupling a connector to the open end of the elongated member, the connector configured to be coupled to a fluid transfer device to transfer fluid into the lumen and out of the plurality of holes.
Clause 14. The method of clause 13, wherein the connector includes a valve body, a rotatable member, and a threaded receptacle.
Clause 15. The method of clause 13 or 14, further comprising: movably coupling a retractable sleeve to the elongated member, the retractable sleeve including a lumen, at least a portion of the elongated member being disposed within the lumen of the retractable sleeve, the retractable sleeve configured to move from a proximal position on the elongated member to a distal position on the elongated member, the retractable sleeve, in the proximal position, not covering the plurality of holes, the retractable sleeve, in the distal position, covering at least a portion of the plurality of holes.
Clause 16. A medical device comprising: an elongated member including a sidewall defining a lumen, the elongated member including a perforated section with a plurality of holes that extend through the sidewall; and a connector configured to connect an open end of the lumen to a fluid transfer device to receive lubricating fluid.
Clause 17. The medical device of clause 16, wherein the elongated member includes a distal end portion and a non-perforated section, the non-perforated section including a proximal end portion of the elongated member, the perforated section being disposed between the distal end portion and the non-perforated section, the perforated section including the plurality of holes.
Clause 18. The medical device of clause 17, wherein the distal end portion includes a solid tip portion defining a closed end of the lumen, the proximal end portion including the open end of the lumen.
Clause 19. The medical device of clause 17, wherein the non-perforated section comprises a majority of a length of the elongated member.
Clause 20. The medical device of clause 16, wherein the elongated member includes a tube member and a protective layer on a surface of the tube member, the plurality of holes extending through the tube member and the protective layer.
Clause 21. The medical device of clause 16, wherein the elongated member includes a guidewire, a biasing member, or a dilator.
Clause 22. The medical device of clause 16, wherein the connector includes a ring member, the connector including a Tuohy Borst connector with a threaded receptacle.
Clause 23. The medical device of clause 16, further comprising: a retractable sleeve including a lumen, at least a portion of the elongated member being disposed within the lumen of the retractable sleeve, the retractable sleeve configured to move from a proximal position on the elongated member to a distal position on the elongated member, the retractable sleeve, in the proximal position, not covering the plurality of holes, the retractable sleeve, in the distal position, covering at least a portion of the plurality of holes.
Clause 24. The medical device of clause 16, further comprising: a device configured to receive the elongated member, the lubricating fluid minimizing a friction between the device and the elongated member.
Clause 25. A medical device comprising: a device including a tube member defining a first lumen and a second lumen, the device including a plurality of holes that extend from the first lumen to the second lumen, the first lumen including a closed end and an open end, the open end configured to receive lubricating fluid from a fluid transfer device; and an elongated member configured to be inserted into the second lumen, the device configured to move lubricating fluid through the first lumen, through the plurality of holes, and into the second lumen.
Clause 26. The medical device of clause 25, wherein the device includes a proximal end portion, the medical device further comprising: a connector configured to be connected to the open end of the first lumen on the proximal end portion of the device.
Clause 27. The medical device of clause 25, wherein the device includes a third lumen and a fourth lumen.
Clause 28. The medical device of clause 25, wherein the elongated member includes a guidewire.
Clause 29. The medical device of clause 25, wherein the first lumen and the second lumen are parallel to each other.
Clause 30. The medical device of clause 25, wherein the first lumen includes an annular lumen that surrounds the second lumen, the second lumen including a linear lumen.
Clause 31. The medical device of clause 25, wherein the device includes an outer sheath, the outer sheath including a sidewall that the first lumen that surrounds the second lumen.
Clause 32. A method comprising: inserting a device into a body of a patient, the device including a lumen; inserting an elongated member into the lumen of the device; and moving lubricating fluid into the lumen of the device to minimize a friction between the device and the elongated member.
Clause 33. The method of clause 32, wherein the lumen is a first lumen, the device including a second lumen, the device including a plurality of holes that extend from the second lumen to the first lumen, the method further comprising: receiving, via the second lumen, the lubricating fluid from a fluid transfer device; and transferring the lubricating fluid from the second lumen to the first lumen via the plurality of holes.
Clause 34. The method of clause 32, wherein the lumen is a first lumen, the elongated member including a sidewall defining a second lumen, the elongated member including a plurality of holes that extend through the sidewall, the method further comprising: receiving, via the second lumen of the elongated member, the lubricating fluid from a fluid transfer device; and transferring the lubricating fluid from the second lumen to the first lumen via the plurality of holes.
Clause 35. The method of clause 34, wherein the elongated member includes a tube member and protective layer on a surface of the tube member, the plurality of holes extending entirely through the tube member and the protective layer.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. A medical device comprising:
an elongated member including a sidewall defining a lumen, the elongated member including a perforated section with a plurality of holes that extend through the sidewall; and
a connector configured to connect an open end of the lumen to a fluid transfer device to receive lubricating fluid.

2. The medical device of claim 1, wherein the elongated member includes a distal end portion and a non-perforated section, the non-perforated section including a proximal end portion of the elongated member, the perforated section being disposed between the distal end portion and the non-perforated section, the perforated section including the plurality of holes.

3. The medical device of claim 2, wherein the distal end portion includes a solid tip portion defining a closed end of the lumen, the proximal end portion including the open end of the lumen.

4. The medical device of claim 2, wherein the non-perforated section comprises a majority of a length of the elongated member.

5. The medical device of claim 1, wherein the elongated member includes a tube member and a protective layer on a surface of the tube member, the plurality of holes extending through the tube member and the protective layer.

6. The medical device of claim 1, wherein the elongated member includes a guidewire, a biasing member, or a dilator.

7. The medical device of claim 1, wherein the connector includes a ring member, the connector including a Tuohy Borst connector with a threaded receptacle.

8. The medical device of claim 1, further comprising:
a retractable sleeve including a lumen, at least a portion of the elongated member being disposed within the lumen of the retractable sleeve, the retractable sleeve configured to move from a proximal position on the elongated member to a distal position on the elongated member, the retractable sleeve, in the proximal position, not covering the plurality of holes, the retractable sleeve, in the distal position, covering at least a portion of the plurality of holes.

9. The medical device of claim 1, further comprising:
a device configured to receive the elongated member, the lubricating fluid minimizing a friction between the device and the elongated member.

10. A medical device comprising:
a device including a tube member defining a first lumen and a second lumen, the device including a plurality of holes that extend from the first lumen to the second lumen, the first lumen including a closed end and an open end, the open end configured to receive lubricating fluid from a fluid transfer device; and
an elongated member configured to be inserted into the second lumen, the device configured to move lubricating fluid through the first lumen, through the plurality of holes, and into the second lumen.

11. The medical device of claim 10, wherein the device includes a proximal end portion, the medical device further comprising:
a connector configured to be connected to the open end of the first lumen on the proximal end portion of the device.

12. The medical device of claim 10, wherein the device includes a third lumen and a fourth lumen.

13. The medical device of claim 10, wherein the elongated member includes a guidewire.

14. The medical device of claim 10, wherein the first lumen and the second lumen are parallel to each other.

15. The medical device of claim 10, wherein the first lumen includes an annular lumen that surrounds the second lumen, the second lumen including a linear lumen.

16. The medical device of claim 10, wherein the device includes an outer sheath, the outer sheath including a sidewall that the first lumen that surrounds the second lumen.

17. A method comprising:
inserting a device into a body of a patient, the device including a lumen;
inserting an elongated member into the lumen of the device; and
moving lubricating fluid into the lumen of the device to minimize a friction between the device and the elongated member.

18. The method of claim 17, wherein the lumen is a first lumen, the device including a second lumen, the device including a plurality of holes that extend from the second lumen to the first lumen, the method further comprising:
receiving, via the second lumen, the lubricating fluid from a fluid transfer device; and
transferring the lubricating fluid from the second lumen to the first lumen via the plurality of holes.

19. The method of claim 17, wherein the lumen is a first lumen, the elongated member including a sidewall defining a second lumen, the elongated member including a plurality of holes that extend through the sidewall, the method further comprising:
receiving, via the second lumen of the elongated member, the lubricating fluid from a fluid transfer device; and
transferring the lubricating fluid from the second lumen to the first lumen via the plurality of holes.

20. The method of claim 19, wherein the elongated member includes a tube member and protective layer on a surface of the tube member, the plurality of holes extending entirely through the tube member and the protective layer.
